(19) United States
(12) Patent Application Publication
(10) Pub. No.: US 2006/0018487 A1
(43) Pub. Date: Jan. 26, 2006

(54) Transducer for sensing body sounds

(76) Inventor: Clive Smith, Englewood, CO (US)

Correspondence Address: Colin P. Abrahams, 5850 Canoga Avenue, Suite 400, Woodland Hills, CA, 91367, US

(21) Appl. No.: 10/964,575

(22) Filed: Oct. 13, 2004

Related U.S. Application Data
(63) Continuation-in-part of application No. 10/747,863, filed on Dec. 23, 2003.
Application No. 10/747,863 is a continuation-in-part of application No. 10/730,750, filed on Dec. 8, 2003.
Application No. 10/730,750 is a continuation of application No. 10/328,768, filed on Dec. 23, 2002, now Pat. No. 6,661,897.
Application No. 10/328,768 is a continuation-in-part of application No. 09/431,717, filed on Oct. 28, 1999, now Pat. No. 6,498,854.

(51) Int. Cl. A61B 7/04; H04R 11/02

(52) U.S. Cl. 381/67; 381/423

(57) ABSTRACT

The structure and method of manufacture of diaphragms for use in electronic body sound transducers is described. The diaphragm is a critical element in shaping the overall amplitude and frequency response of such transducers. Various embodiments of novel diaphragms are described, each of which offers particular advantages in shaping the transducer response and the mechanical or electrical characteristics of the diaphragms. Manufacturing steps for producing such diaphragms are also disclosed. The diaphragms can be used in any electronic body sound transducer, particularly capacitive, magnetic or optical transducers. Such diaphragms and transducers are applicable to the sensing of body sounds in electronic stethoscopes.

### CROSS REFERENCE TO RELATED APPLICATIONS

This application is a continuation-in-part of U.S. patent application Ser. No. 10/747,863 filed Dec. 23, 2003, which is a continuation-in-part of U.S. patent application Ser. No. 10/730,750 filed Dec. 8, 2003, which is a continuation of U.S. patent application Ser. No. 10/328,768 filed Dec. 23, 2002, now U.S. Pat. No. 6,661,897, which is a continuation in part of U.S. patent application Ser. No. 09/431,717 filed Oct. 28, 1999, now U.S. Pat. No. 6,498,854, all of which are incorporated herein by reference in their entirety.

### BACKGROUND OF THE INVENTION

The present invention relates to transducers for sensing body sounds, and more specifically to the diaphragms used on such transducers. This invention applies particularly to body sound transducers used in electronic stethoscopes.

Historically, stethoscopes have been mechanical devices that rely on sound waves traveling via air tubes to the listener's ears. Such devices lack sensitivity and do not particularly amplify body sounds. More recently, electronic stethoscopes have been developed that provide substantial amplification.

In the aforementioned application, capacitive, magnetic and optical transducers are disclosed. These transducers are highly sensitive. Specifically, the dynamic characteristics of the diaphragm are far more critical in these transducers. This affords the new possibility of finely controlling the characteristics and response of the transducer by designing the diaphragm to respond with particular dynamics. The present invention thus discloses new diaphragm structures and methods of manufacture that provide for the control of acoustic dynamics of the aforementioned electronic body sounds transducers.

### SUMMARY OF THE INVENTION

Electronic body sound transducers, and electronic stethoscope sensors in particular, are highly sensitive devices. Specifically, the capacitive, magnetic and optical transducers disclosed in the aforementioned application are highly sensitive transducers in which the diaphragm dynamics can have a large influence on transducer response. The present invention discloses specific diaphragm structures and methods for manufacturing diaphragms so that the sensitivity of the transducers to diaphragm characteristics can be exploited, to achieve specific and desirable transducer response. This is a surprising result, in that it is normally desirable in any system to reduce sensitivity to the characteristics of any one element. Instead, the present invention accepts that sensitivity to diaphragm dynamics is part of the transducer performance, and should be exploited as a method of influencing transducer performance by making changes in the structure and manufacture of the diaphragm.

In the specific case of the capacitive transducer, further changes in the diaphragm can be used to adjust transducer dynamics. Since the diaphragm is both a mechanical and electrical element, the diaphragm can be designed such that mechanical dynamics are fine-tuned, and electrical characteristics are fine-tuned, to achieve the overall performance.

Mechanical dynamics of a the diaphragm can be adjusted in one or more of the following ways:

a. Multiple, different uniform-thickness diaphragms may be provided, such that diaphragms can be interchanged, each diaphragm being a different thickness. Thus transducer response can be adjusted by providing a mechanism for changing diaphragms, along with a selection of two or more diaphragms that have difference characteristics.

b. The diaphragm can be of non-uniform thickness and/or contoured across its surface, such that the mechanical stiffness and vibrational characteristics are fine-tuned.

These innovations are in contrast to the prior art, in which transducers, and stethoscopes in particular, are offered with diaphragms of only one thickness, and the diaphragms are uniform in thickness across their entire area.

Electrical characteristics of a diaphragm can be adjusted in the case of the capacitive transducer by having a non-uniform thickness across the diaphragm's surface in the capacitive space between the diaphragm and the capacitive plate. Capacitance is a function of the gap between the plates of a capacitor. By manufacturing a diaphragm with varying thickness, the gap between the capacitive plates can be adjusted according to position on the diaphragm. For example, the center of the diaphragm might be thinner such that the capacitance is lower at the center of the diaphragm than at the outer edges. This can affect the frequency response and sensitivity of the transducer as a function of location on the diaphragm itself. Thus the transducer might be made less sensitive to vibration at the center of the diaphragm, and more sensitive to vibration at the outer radii.

Finally, this invention includes steps for manufacturing diaphragms with varying thickness and dynamics. Specifically, the invention discloses the process of fabricating diaphragms using injection-molding of plastics. The prior art typically uses flat sheets of plastic or glass-epoxy which are then simply cut into round diaphragms. Injection molding affords the possibility of being able to tightly control the entire shape of the diaphragm, beyond it being a simple flat disc.

### BRIEF DESCRIPTION OF THE DRAWINGS

FIG. 1 shows an acoustic to electrical transducer wherein the transducer characteristics can be modified by attaching one of a selection of diaphragms.

FIG. 2 shows various contours or thickness profiles of diaphragms.

FIG. 3 shows raised dimples on the outer circumference of a diaphragm to allow free motion of most of the diaphragm circumference, rather than being clamped around the entire circumference. Also shown is an attachment means with dimples to allow for free movement of the diaphragm circumference.

FIG. 4 shows capacitive transducers wherein the gap between the diaphragm and fixed plate change across the surface area of the diaphragm, due either to a variable height profile across the diaphragm, the fixed plate, or both.

FIG. 5 shows a diaphragm with regions which have differing thickness, such regions not necessarily being symmetrically placed around the central axis of a circular diaphragm.

FIG. 6 shows two manufacturing processes for producing diaphragms with contoured or variable thickness and/or height profiles—injection molding and thermoforming plastic film or sheets.

FIG. 7 shows the fabrication and structure of a capacitive diaphragm with conductive and insulating layers.

### DETAILED DESCRIPTION OF THE PREFERRED EMBODIMENT

This invention discloses electronic body sound transducer diaphragms, and their manufacture. In all the drawings, it is to be understood that the diaphragms are shown in cross-sectional views, to show thickness profiles. The diaphragms are typically circular, although other shapes are possible.

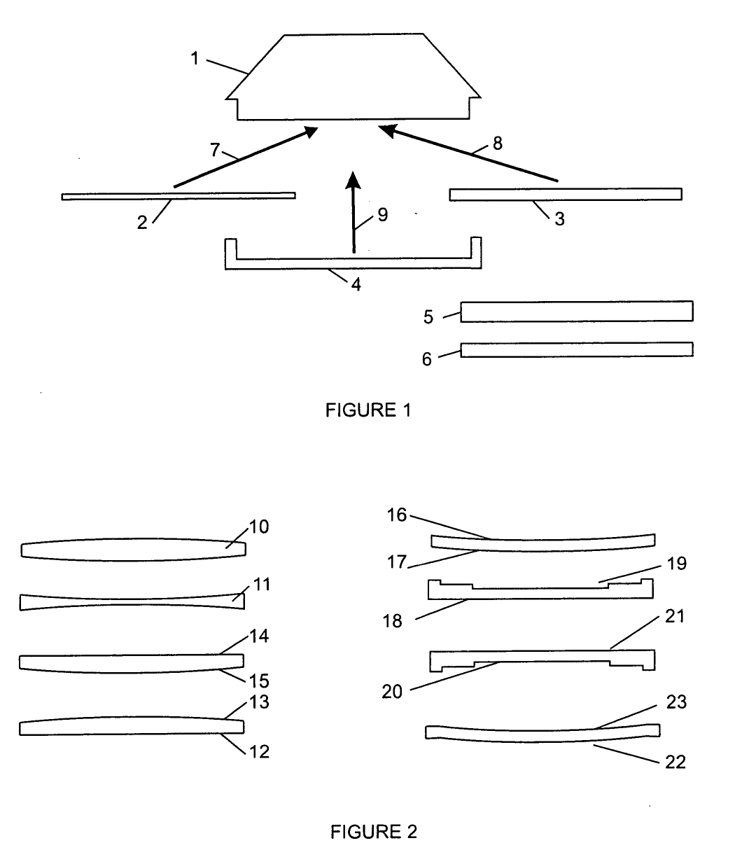

FIG. 1 shows one preferred embodiment of the invention. A body sounds transducer is shown in a housing 1, with a retainer means 4 for attaching the diaphragm to the transducer housing 1. Two diaphragms are shown, one thin diaphragm 2, one thicker diaphragm 3. The transducer response can be adjusted by following the steps 7 or 8 of attaching diaphragm 2 or attaching diaphragm 3 respectively, followed by the step 9 of attaching attachment means 4 to secure the respective diaphragm to the housing 1. Also shown in FIG. 1 is the additional possibility of providing more than 2 diaphragms, shown schematically as diaphragms 5 and 6. The invention thus includes two or more different diaphragms, each with different mechanical and/or electrical characteristics such that attachment of one or other diaphragm results in a different transducer response to vibration.

In one embodiment, the thinner diaphragm is more flexible, providing greater sensitivity, especially to frequencies below about 50 Hz to 100 Hz. The thicker diaphragm is less sensitive, and will tend to attenuate signals of low frequency, resulting in less sensitivity to very low or sub-sonic frequencies. In another embodiment, each of the diaphragms in the set are fabricated with different materials, such that one material is stiffer than the other(s). A stiffer material provides a similar function to increasing the thickness of the material i.e. less sensitivity to low frequencies. Conversely, a more flexible material provides for increased sensitivity, especially at low frequencies.

Referring to the embodiment of the invention shown in FIG. 1, two or more diaphragms are provided with different thickness. One diaphragm is nominally 15 mils in thickness for higher sensitivity, and the second is 20 mils thick. An alternative set can include 2 or more of the following thicknesses—10 mil, 12 mil, 15 mil, 16 mil, 20 mil, 25 mil. These thicknesses have been found to be suitable for use in stethoscopes. It is to be noted that these are nominal thicknesses and the invention allows for nominal tolerance variations in these thicknesses. Typical tolerance is plus or minus 0.5 to 2 mils.

Another method for adjusting the dynamics of the diaphragm is to vary the force applied by the attachment means 4, such that the force acting on the outer circumference of the diaphragm (2, 3, 5, 6) by the attachment means 4 or the transducer housing 1 can be varied. By providing this adjustable force means to the operator, the operator can then control the stiffness and tension of the diaphragm. It can also be advantageous to shape the diaphragm circumference to interact with the attachment means to achieve adjustable stiffness or tension. In one embodiment, the outer circumference of the diaphragm is sloped so that additional pressure on the diaphragm causes it to bow and thereby change its acoustic response. In the case where force is used to adjust diaphragm response, only one diaphragm might be needed in the invention, rather than multiple diaphragms.

The invention shown in FIG. 1 should also be understood to include any interchangeable diaphragm system wherein the dynamics and acoustic characteristics of an electronic vibration transducer are adjusted by interchangeable diaphragms, each diaphragm producing a specific performance characteristic. More specifically, it can also be any of the capacitive, magnetic, or optical sensing methods disclosed in the aforementioned applications. In the capacitive embodiment, the diaphragm includes a conductive layer or surface that is connected as part of a capacitive plate of the capacitive transducer.

FIG. 2 shows a number of embodiments of the invention in which the diaphragm has a non-uniform cross-section with changing thickness and/or contour. The contour can be a stepped surface, curved surface, or other profile which is not uniformly flat on one or both sides of the diaphragm. FIG. 2 shows cross-sectional side views. The cross-section is designed to control the stiffness and displacement of various regions of the diaphragm, such that the overall dynamics of the diaphragm achieve a desired acoustic response. Such dynamics include shaping the amplitude sensitivity, and shaping frequency response. Specifically, a thinner outer radius 10 allows for greater displacement of the inner region of the diaphragm as whole. A thinner inner region 11 allows for the center of the diaphragm to be displaced to a greater extent than the outer regions. Another interesting benefit of the variable-thickness diaphragm is that vibrations emanating from the outer attachment means can be damped, so that such vibrations of the attachment or casing do not reach the inner surface of the diaphragm, thereby attenuating outside sound or noise. Other embodiments shown in FIG. 2 include flat outer surface 12 and curved inner surface 13; curved outer surface 15 with flat inner surface 14; curved parallel outer and inner layers 16 and 17; flat outer layer 18 and stepped inner layer 19; stepped outer layer 20 and flat inner layer 21. The two inventive characteristics common to these diaphragms are (a) one or more surface is not flat and/or (b) the thickness of the diaphragm is not necessarily constant across the entire area of the diaphragm.

One embodiment shown in FIG. 2 shows a diaphragm with curved surfaces 22 and 23. Such a surface provides the benefit of controlling or presetting the tension of the diaphragm prior to displacement due to placing it against the skin. This is a pre-compensation for the displacement that occurs when the diaphragm is pressed against the body.

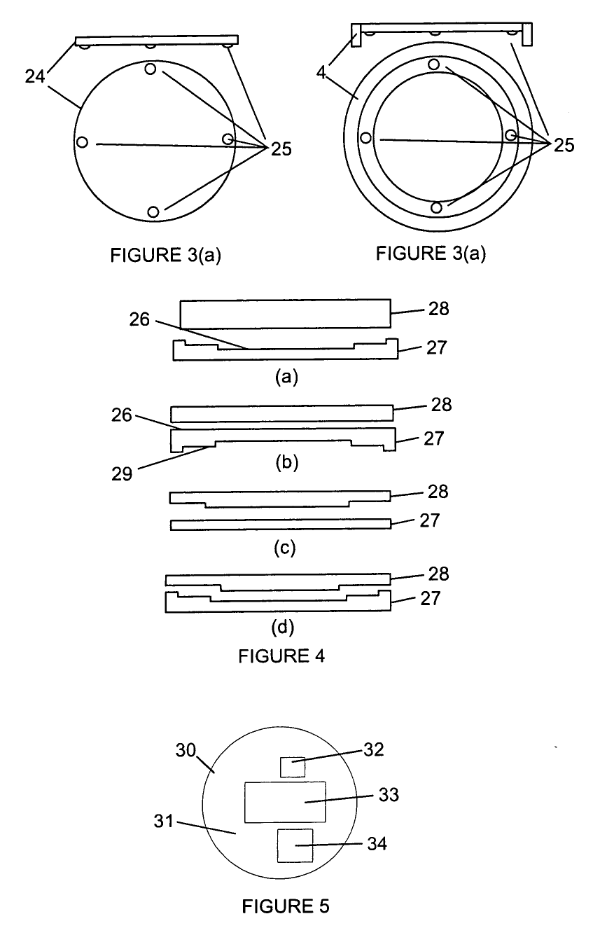

FIG. 3(a) shows a diaphragm 24 in which the outer rim is shaped for attachment to the transducer housing. It is beneficial for the diaphragm to float as freely as possible. Fixing the outer rim around the entire circumference of the diaphragm can result in undesirable restriction of the diaphragm. The figure shows dimples or ridges 25 that are created on the diaphragm, such that a space is provided for most of the diaphragm circumference to move freely, since the dimples prevent the attachment means (FIG. 1, element 4) from clamping the entire circumference. FIG. 3(b) shows an attachment means 4, wherein ridges or dimples 25 are placed on the surface which applies a securing force to the diaphragm when attached to the transducer housing 1 of FIG. 1. This embodiment also allows the circumference of the diaphragm to move more freely than if it were uniformly clamped around the circumference.

FIG. 4 shows a diaphragm specifically suited to use in the capacitive transducer. A capacitive sensor is formed by a fixed conductive capacitive plate 28 placed at a distance from a moveable diaphragm 27, the diaphragm 27 having a conductive capacitive layer, such that when the diaphragm is displaced, the gap between the capacitive plates is modulated by the sound or vibration thereby varying capacitance as a function of acoustic or vibratory signal. The two plates are then connected to a circuit which can transducer the capacitance change into an electrical signal. Referring to FIG. 4(a), The inner surface 26 of the diaphragm 27, on which the conductive capacitive surface exists, is not uniformly flat across the capacitive plate surface. Instead, there are raised or lowered surface areas across the diaphragm. When placed in a capacitive transducer with a capacitive plate 28, the raised areas result in a reduced capacitive gap, and the lower is surfaces result in a larger gap in the capacitance for those areas. The result is that the capacitance is controlled according to specific regions of the diaphragm surface. This allows for very fine tuning of the electrical response to different regions of the diaphragm. For example, a lowered central portion of the diaphragm with a larger gap results in a lower capacitance per unit area for the central portion of the diaphragm.

FIG. 4(b) shows a capacitive diaphragm 27 with a flat inner surface with flat conductive layer 26, and a variable thickness due to changes in the outer surface 29.

FIG. 4 (c) shows a capacitive sensor structure in which the fixed conductive plate 28 has a variable surface height, and the diaphragm 27 is flat. In this embodiment, the capacitance is controlled by the plate surface contour. Note that the figure shows a stepped contour, but a continuously variable surface contour is an alternative embodiment.

FIG. 4(d) shows a fixed conductive plate 28 and a diaphragm 27 wherein both capacitive surfaces are contoured. In this embodiment, the thickness variations in the diaphragm can be controlled for mechanical characteristics, and the capacitive characteristics controlled separately by the contour of the fixed capacitive plate. This allows for independently controlling mechanical and capacitive/electrical characteristics.

The fixed capacitive surfaces shown in FIGS. 4(a) to (d) can be implemented as a fixed conductive layer on a substrate, or solid metal. The fixed capacitive surface can also be implemented on a semiconductor device wherein the surface is fabricated in silicon, poly-silicon or metal on a substrate, and the diaphragm is placed over the fixed surface to form a micromechanical semiconductor capacitive transducer.

FIG. 5 shows a diaphragm 30 with different regions 31 through 34, wherein each region has a different thickness or height. This figure shows that the thickness or height variation are not necessarily symmetrical, and the invention provides for asymmetrical thickness and/or height profiles that are not symmetrical with respect to the central axis of a symmetrical diaphragm. Note that while four regions are shown, there can be two or more such regions.

The invention discloses a novel method of fabricating diaphragms for body sound or stethoscope transducers. In the prior art, diaphragms are fabricated by cutting fixed-thickness sheets of plastic or glass epoxy. In this invention, the diaphragms can also be fabricated by injection molding plastic to form the particular shape and thickness required. The injection molding process can also include the step of using more than one material in the molding process, such as two plastics with different stiffness characteristics, such as a stiff material on the outer radius, and a softer material on the inner radius. The plastics used for such molding include polycarbonate, nylon, ABS plastic, glass-filled polycarbonate, glass-filled nylon, and soft materials such as silicone or PVC. This is not an exhaustive lists and it is to be understood that any plastic suitable for injection-molding can be used for fabricating the diaphragms.

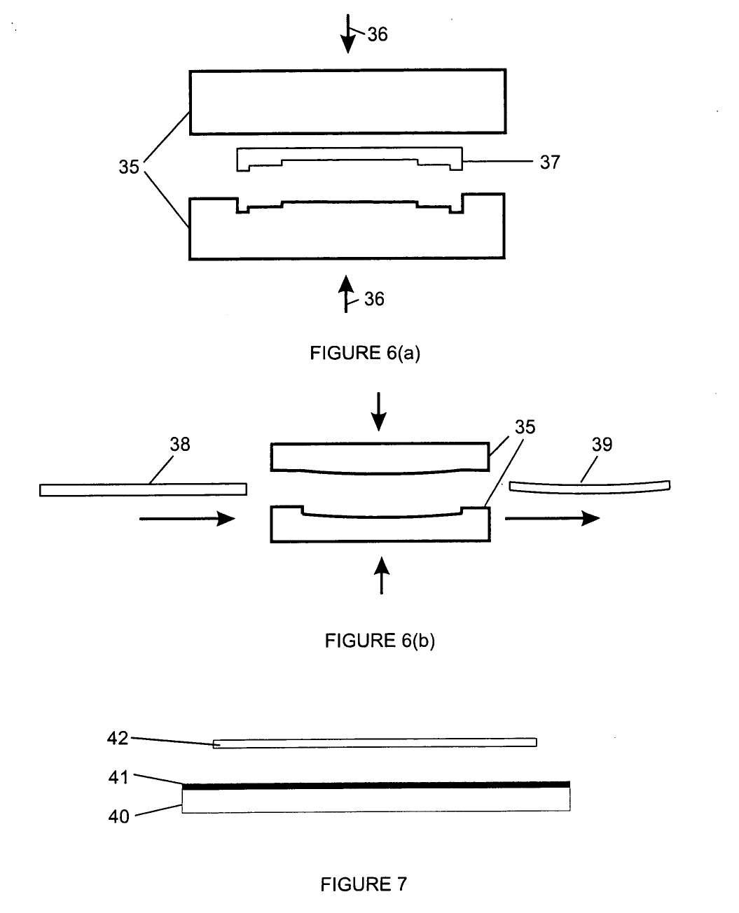

FIG. 6(a) shows the embodiment of an injection-molded manufacturing process. A tool 35, shaped to produce the desired shape is pressed 36 under high temperature to produce a molded diaphragm 37. One advantage of such a process is that any shape or diaphragm profile can be produced with very high precision, allowing for very tightly controlled acoustic response. Any and all of the profiles shown in this invention can be produced by this process, including outer circumference dimples, and multiple materials.

Another method of manufacturing the diaphragms disclosed in this invention is by thermoforming—pressing fixed-thickness plastic films under high temperature, to conform to the desired shape. This method is especially applicable where the diaphragm is of somewhat constant thickness, but the contour or outer dimples must be added to the previously-flat film. FIG. 6(b) shows this process. Flat film material 38 is inserted into the tool 35, which thermoforms the film, changing its contour. The result is a shaped diaphragm 39.

FIG. 7 shows one process of manufacturing a capacitive diaphragm. Substrate 40 which can be flat or contoured as described above, has a conductive layer 41 applied to its surface. Methods for applying this layer include silkscreening conductive ink, spraying, or vapor deposition. An insulator 42 is then applied over the conductive layer. This insulator can be an adhesive insulating sticker, such as Teflon, adhered to the diaphragm, or an insulating material applied to the surface, such as a dielectric liquid that is applied and cured. The insulating layer need not cover the entire conductive surface of the diaphragm, and in one embodiment some of the conductive layer is exposed to allow for electrical contact between the layer and the capacitive sensing electrical circuit.

1. An acoustic to electrical transducer for detecting body sounds, comprising:
a transducer placed inside a housing;
a diaphragm that forms the movable sensor element of the transducer mechanism;
a means for attaching the diaphragm to the housing; Two or more diaphragms, each diaphragm having unique mechanical or electrical characteristics;
wherein the transducer amplitude and frequency response can be modified by selectively attaching one of many diaphragms to the housing.

2. The diaphragms in claim 1 wherein the unique mechanical characteristic of each diaphragm is the thickness of each diaphragm.

3. The diaphragm in claim 3 wherein the thickness of each diaphragm is selected from one of the following: 10 mils, 12 mils, 15 mils, 16 mils, 20 mils, 25 mils.

4. The diaphragm in claim 1 wherein the unique mechanical characteristic of each diaphragm is the surface contour of each diaphragm.

5. The diaphragm in claim 1 wherein the diaphragm has raised surfaces around the circumference of the diaphragm, in the area of contact with the housing or attachment means.

6. An acoustic to electrical transducer for detecting body sounds, comprising a capacitive transducer with a first plate of the capacitive sensor being a fixed plate, and the second plate being a conductive layer of a movable diaphragm which contacts the body; wherein the gap between the first and second capacitive plates varies as a function of the profiles of each plate, such that the capacitance per unit area between the diaphragm and the fixed plate varies across the area of the diaphragm-plate gap as a function of said profiles.

7. A diaphragm for use in an acoustic to electrical transducer for sensing body sounds, the diaphragm being fabricated by injection-molding plastic to form a contoured diaphragm surface.

8. A diaphragm for use in an acoustic to electrical transducer for sensing body sounds, the diaphragm being fabricated by thermoforming flat plastic sheets, to form a contoured diaphragm surface.
